(19) United States
(12) Patent Application Publication
(10) Pub. No.: US 2012/0165934 A1
(43) Pub. Date: Jun. 28, 2012

(54) COLLAPSIBLE, FORM-STABLE, FILLABLE BREAST PROSTHESIS

(75) Inventor: David J. Schuessler, Ventura, CA (US)

(73) Assignee: Allergan, Inc., Irvine, CA (US)

(21) Appl. No.: 13/327,038

(22) Filed: Dec. 15, 2011

Related U.S. Application Data
(60) Provisional application No. 61/423,501, filed on Dec. 15, 2010.

(51) Int. Cl. A61F 2/12

(52) U.S. Cl. 623/8

(57) ABSTRACT

Provided is a flexible, collapsible, implantable breast prosthesis which is form stable such that it can be inserted through a relatively small incision and then will regain its configuration and maintain its shape within the breast. The prosthesis may include an anterior portion including a closed-cell silicone foam.

This application claims priority to, and the benefit of, U.S. Provisional Patent Application No. 61/423,501, filed on Dec. 15, 2010, the entire disclosure of which is incorporated herein by this specific reference.

The present invention generally relates to implantable prostheses, and more specifically relates to collapsible, form-stable breast prostheses.

Implantable prostheses are commonly used to replace or augment body tissue. For example, an implantable prosthesis can be used to fill the void caused by removal of the mammary gland and surrounding tissue in the case of breast cancer. Also, it is well known that implantable prostheses are also used to augment breasts for aesthetic reasons.

Various issues may arise from the placement of prosthesis within a breast cavity. For example, it may be a challenge to insert an implant into the cavity via a small incision in the breast. Also, the form, or shape and structure, of the prosthesis may be distorted and need adjustment while in the breast cavity. Furthermore, capsular contracture may occur around an implant, causing discomfort and pain.

Cox, Jr., U.S. Pat. No. 4,944,750 discloses an implantable prosthesis including an envelope comprising an inner wall, and outer wall and a foam layer between the walls in adherent surface-to-surface contact. The foam is described as being resiliently deformable to resist sharp folding of the walls and to resist relative in-plane motion of the walls.

Schuessler et al., U.S. patent application Ser. No. 12/275,111, filed on Nov. 20, 2008, commonly owned herewith, disclosed an insertable preformed sizer for breast implant that regains its form after deformation and insertion into a cavity formed within breast tissue.

The current invention provides an improved prosthesis, for example, an improved fillable breast implant.

### SUMMARY

Accordingly, the present invention relates to a flexible, collapsible prosthesis, hereinafter sometimes referred to as simply “implant”, which is implantable in an unfilled condition and which is essentially form stable. The prosthesis is structured such that it can be inserted through a relatively small incision into a breast cavity, and then will substantially regain its configuration and substantially maintain its shape once within the body.

In one aspect, the invention provides a breast prosthesis, or breast implant, comprising an elastomeric shell having an expanded configuration defining a fillable cavity. The shell comprises an anterior portion, for example, a convex, rounded or anatomically curved anterior portion, and a posterior portion, for example a substantially planar portion, substantially opposing the anterior portion. The fillable cavity is defined between the posterior portion and the anterior portion when the implant is in its expanded state.

Advantageously, at least one of the anterior portion and the posterior portion, typically the anterior portion, includes a form-stabilizing material that is effective to maintain the expanded state of the implant, and moreover, will maintain a desired shape of the implant, for example, a rounded anterior and a substantially planar posterior, when the implant is located in the body.

The form-stabilizing material preferably comprises a collapsible, resilient material that will function to maintain a desired shape of the implant while in the body. The form-stabilizing member comprises, for example, a resilient, elastomeric cellular material, hereinafter referred to as a foam, or foam-like material.

In one embodiment, the form-stabilizing portion does not entirely circumscribe the cavity. For example, the form-stabilizing portion may be disposed on, or make up a portion of, the anterior portion of the shell, the posterior portion of the shell, or a combination of these two, without being disposed on or making up the entirety of both of the anterior portion and posterior portion. In other words, the form stabilizing material does not entirely encompass or surround the cavity. This feature may facilitate folding or rolling of the implant for insertion into a relatively small incision into a breast cavity, without compromising the form stabilizing effect.

In a specific embodiment, the form-stabilizing portion forms at least one of an inner surface and an outer surface of the anterior portion. The form-stabilizing portion may have a curved or anatomical shape.

The shell may include a port or opening to facilitate filling of the cavity. The port or opening may be located on the posterior portion of the implant. Before or after the prosthesis is implanted, the cavity may be filled with a suitable liquid, for example, a saline or silicone gel, like a conventional fillable implant.

In some embodiments, the form-stabilizing portion is a porous material having a relatively low density relative to silicone gel or saline solution. Further, the form-stabilizing portion preferably is a highly resilient material that will regain its shape after being collapsed and inserted through a small incision into a breast cavity.

In some embodiments, the form-stabilizing portion has at least one major surface of which is substantially liquid impermeable. Particularly in embodiments of the invention in which the form-stabilizing portion has a surface interfacing any of the fillable cavity, the form-stabilizing portion is structured that it will not take up or absorb any of the filling used in the implant.

In one embodiment, the form stabilizing portion comprises a foam member, for example, a silicone foam member, and may be substantially entirely closed-cell foam. In an especially advantageous embodiment, the foam member is a closed-cell silicone foam.

In yet another embodiment, the form stabilizing portion comprises a foam member, for example, a silicone foam member, and may be substantially entirely closed-cell foam having a non-porous “skin” side interfacing the prosthesis cavity, and a porous, textured side for interfacing the patient's tissue when implanted. In an especially advantageous embodiment, the foam member is a closed-cell, self-skinning, silicone foam.

In many embodiments, the implant is collapsible for facilitating implantation in an unfilled state, through a small incision. Preferably, the foam portion making up at least a portion of the anterior surface of the prosthesis is sufficiently elastic and resilient such that it will spring back into the desired form after it has been implanted. In other words, the implant is substantially form-stable. In certain embodiments, the foam portion is a closed-cell foam (as distinguished from an open-celled foam. Closed air cavities in the foam make for a lower-density, lighter implant relative to a conventional, identically sized and shaped implant that may include open celled foam.

In another aspect of the invention, a prosthesis is provided having a form stabilizing element. Advantageously, the form stabilizing element may comprise a flexible, resilient material that substantially maintains a shape after implantation. For example, in some embodiments, the form-stabilizing portion is a foam material, for example a layer of biocompatible foam material, for example a closed-cell foam material.

In some embodiments, the form stabilizing element comprises a foam layer extending only across an anterior portion of the shell. For example, in these embodiments, the form-stabilizing portion is not present on the posterior portion of the shell. Interior surfaces of the posterior and anterior portions define a cavity of the shell. The shell further includes a port or opening, for example through the posterior portion of the shell, which is useful for filling the cavity with saline solution or a suitable gel, for example, silicone gel.

Each and every feature described herein, and each and every combination of two or more of such features, is included within the scope of the present invention provided that the features included in such a combination are not mutually inconsistent.

### BRIEF DESCRIPTION OF THE DRAWINGS

The present invention may be more clearly understood and certain aspects and advantages thereof better appreciated with reference to the following Detailed Description when considered with the accompanying Drawings of which:

FIG. 1 is side cross-section view of an embodiment of the current invention showing the anterior and posterior sides of the prosthesis and the materials used for construction of the prosthesis; in this embodiment, the form-stabilizing portion is the inner or interior layer of the anterior side;

FIG. 2 is a side cross-section view of an embodiment of the current invention showing a different prosthesis shape than the one featured in FIG. 1 and the foam layer as the outer or exterior layer of the anterior side; and

FIG. 3 is an illustration of closed and open cell foam structures.

FIG. 4 is a cross sectional view of another embodiment of the invention.

### DETAILED DESCRIPTION

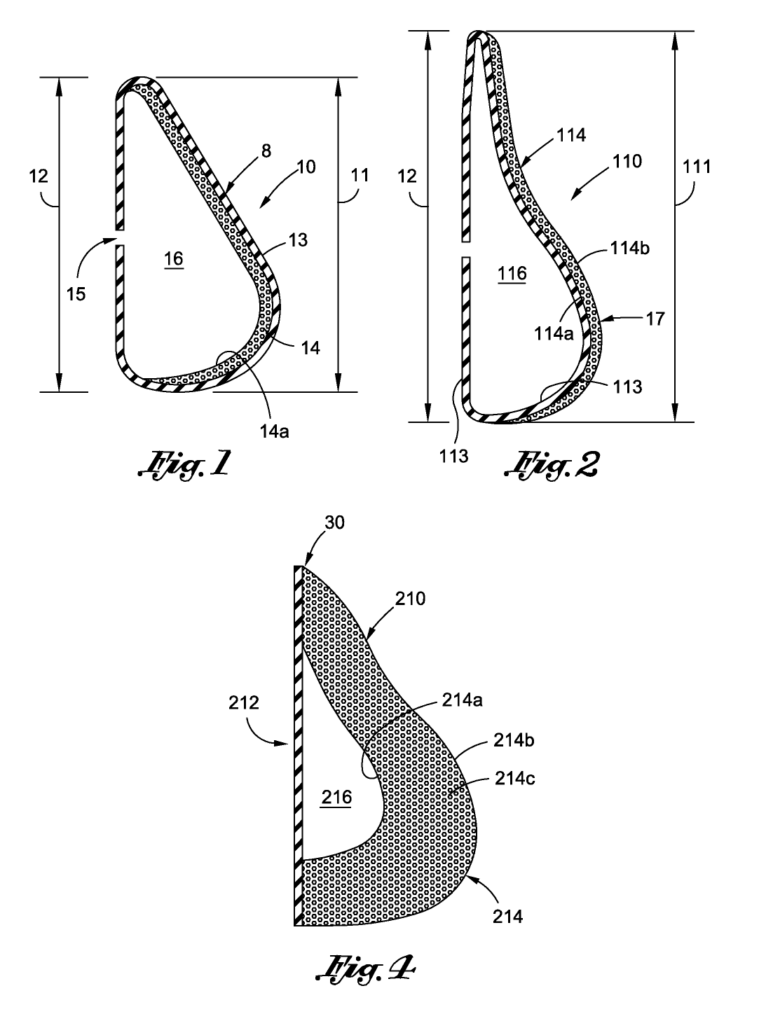

Turning now to FIG. 1, an implantable breast prosthesis 10 is provided.

In a broad aspect of the invention, the prosthesis 10 comprises a hollow, collapsible shell 8 comprising, or made up of, an anterior portion 11 and a substantially opposing posterior portion 12. A cavity 16, for example, a fluid-fillable cavity, is defined between the anterior portion 11 and the posterior portion 12. A form-stabilizing portion, to be described hereinafter, is disposed on or makes up at least a part of, at least one of the anterior portion 11 and the posterior portion 12. In some embodiments, the form-stabilizing portion comprises a material and/or structure that provides a resiliency to the shell 8 such that the shell 8 will substantially entirely regain its configuration in a body cavity after being inserted in a collapsed or folded configuration into the body cavity, for example, through a relatively small incision, when the prosthesis 10 is in an unfilled state.

In a particularly advantageous embodiment, the prosthesis 10 is substantially entirely comprised of silicone.

In a specific embodiment of the invention, the anterior portion 11 has a rounded or anatomical shape conforming to a conventional breast implant, and the posterior portion is generally planar. The anterior portion 11 may include a layer of elastomeric material 13, and a layer of a substantially closed-cell, resilient material, for example, a foam material 14. The elastomeric material 13 may extend beyond the anterior portion 11 and may form the posterior portion 12 of the prosthesis 10, as shown. The foam material 14 may not entirely circumscribe the cavity 16, but may define only a portion of a surface defining the cavity 16, as shown. In this embodiment, the foam material 14 may include a non-porous side 14a, for example a smooth, non-liquid permeable skin side, interfacing the cavity 16. The foam material may be a closed-cell foam.

In some embodiments, the foam material 14 may be disposed on, or form at least a part of, the posterior portion 12 of the prosthesis 10. In yet other embodiments, not shown, the foam material 14 may be located on a perimeter of the prosthesis 10, for example, circumscribing the prosthesis along a juncture of the anterior portion 11 and the posterior portion 12.

In the shown embodiment, the prosthesis 10 also includes a fill port 15, or other suitable valve or opening, to enable filling of the cavity 16 with saline, silicone gel, or other suitable liquid after implantation of the prosthesis 10.

Turning now to FIG. 2, a prosthesis 110 is shown which is substantially identical to prosthesis 10, with like parts being represented by like reference numerals increased by 100, with a difference being that prosthesis 110 comprises anterior portion 111 comprising an outer layer of the foam material 114.

Prosthesis 110 may also include a layer 113 of elastomer material forming a surface defining cavity 116. Layer 113 may be a separately molded component of prosthesis 110 with foam material 114 adhered or otherwise coupled thereto. Alternatively, layer 113 may be an integrally molded part of foam material 114, for example, layer 113 may be a skin layer, e.g. a smooth, impermeable surface or portion 114a, of the foam material 114 itself, which defines at least a portion of cavity 116.

In this shown embodiment, the foam material 114 defines an outer surface of the prosthesis 110, that is, a surface which interfaces a patient's tissue upon implantation of prosthesis 110. Thus, the foam material 114 may be structured to promote healthy tissue contact. For example, the outer surface of the prosthesis 110 may be a textured surface 114b, for example, a porous or pitted or surface, or otherwise structured to be effective to facilitate or promote healthy tissue adhesion and/or tissue ingrowth. The textured surface 114b may be disposed on at least a portion of the outer surface of the prosthesis 110, for example, on an anterior forward-facing surface of the prosthesis 110.

Surface textured breast implants has been shown to reduce capsular contracture when compared to what are known as “smooth” surface implants. Capsular contracture is a complication that arises from the body's response of forming a capsule of tissue, primarily collagen fibers, around an implanted object, such as a breast implant. The tightening of the capsule of tissue around a breast implant can be discomforting or even extremely painful, and can cause distortion of the appearance of the augmented or reconstructed breast.

In some embodiments of the invention, the foam material, for example, foam material 114 in FIG. 2, is a self-skinning foam. For example, the self-skinning foam 114 comprises a non-porous, skin-side 114a and an textured, porous side 114b. Between the skin side 114a and the porous side 114b, the foam material 114 may be a substantially closed-cell foam, as defined elsewhere herein. It is known to those of skill in the art that self-skinning foam refers to foam material which, during the polymerization process, is able to form its own outer skin or cover that is generally smooth and non-porous. Typically, a self-skinning foam, also sometimes known as integral skinning foam, has a high-density skin and a low-density (porous) core. Processes for making self-skinning foams are known to those of skill in the art, and can be formed in an open mold process or a closed mold process. In the open mold process, two reactive components (e.g. base and catalyst) are mixed and poured into an open mold. The mold is then closed and the mixture is allowed to expand in the mold and cure. In a closed mold process, more commonly known as reaction injection molding (RIM), the mixed reactive components are into a closed mold under high pressures.

In some embodiments of the invention, a self-skinning foam may make up at least some of the anterior portion (or alternatively or additionally, at least some of the posterior portion) of the shell, such that the “skin” of the foam interfaces and defines a boundary of the cavity. An open-celled portion of the self-skinning foam defines at least a portion of the exterior surface of the implant, such that it provides a textured surface for promoting tissue in-growth. In this embodiment, there may be no need for the anterior portion 11 to include a separate elastomeric material as the foam skin itself provides a barrier to absorption of saline or other liquid filling the cavity and advantageously also provides a textured surface on the exterior of the implant.

All materials used for the current invention are biocompatible, in the ordinary sense of the word, meaning the material that is inert, nontoxic or otherwise not harmful when implanted in a patient. One material that may be used for forming the prostheses in accordance with the invention, is silicone elastomer, for example, a material that may be used as the foam material is a silicone foam.

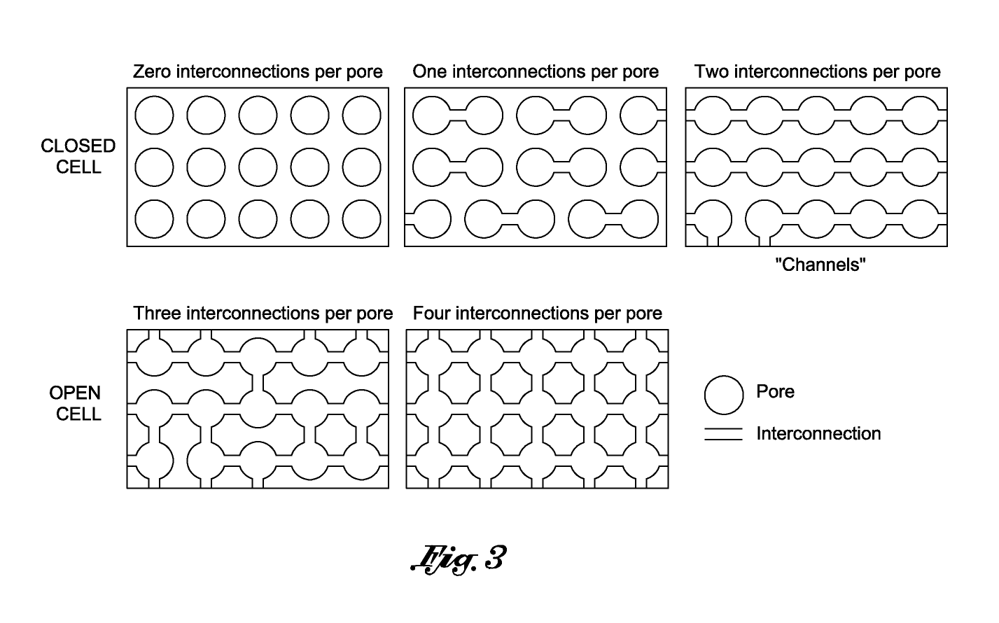

In some embodiments, the foam material comprises closed cell foam. Generally, closed cell foam in accordance with the present description is characterized as having pores with two or fewer channels or interconnections per pore, as opposed to open cell foam, which has three or more channels of interconnections per pore (see FIG. 3). Preferably, the closed cell foam used in the present invention is one which has substantially no channels interconnections between pores.

The closed cell foam may be a foam with a pore size in a range, for example, of between about 10 micron to about 1000 micron, for example, between about 200 micron and about 800 micron, for example, between about 400 micron and about 600 micron. As mentioned hereinabove, the foam may comprise a silicone elastomer or other suitable biocompatible material, and may be soft and compressible to simulate natural human breast tissue. In addition, the foam may have an elongation (from about 100% to about 800%, for example, from about 200% to about 700%, for example, from about 300% to about 600%) to provide the flexibility and resiliency needed for collapsing. The foam may have a low compression set, for example, in the range of less than about 5%, so that it maintains its intended shape after collapsing or being under continuous pressure or compression.

In the current invention, because much of the implant remains hollow prior to implantation, the prosthesis can be collapsed, folded, and/or compressed into a shape small enough to be inserted into a patient's breast cavity through a relatively small incision. After the prosthesis has been inserted inside the breast cavity, the resilient materials comprising the prosthesis allow it to recover, or at least substantially recover, its original, full-sized form within the constraints of the surrounding breast tissue. Moreover, the foam layer of the anterior side contributes to maintenance of the full-sized form of the prosthesis by providing more stability.

The thickness of the foam layer may be between about 1 mm and about 40 mm, depending on the desired size of the whole prosthesis. For example, the foam layer may be at least about 5 mm up to about 30 mm. Also, the foam layer need not be of uniform thickness throughout.

After insertion into a breast cavity, any liquid or gel filler commonly used as an implant filler, such as saline or silicone gel, can be introduced into the cavity. For example, the cavity can be accessed by injecting the filling material into the cavity by means of an opening having a self-sealing septum, for example, on the posterior side of the prosthesis. In some embodiments, introduction of an implant filler into the cavity is not necessary for the prosthesis to regain its full-sized form after being collapsed, folded, and/or compressed; in some embodiments, the resilient nature of the materials comprising the prosthesis (particularly the foam material) may be sufficient to restore or substantially restore the prosthesis to its full-sized form while in the body.

The opening on the posterior side of the prosthesis may be sealed with a penetrable, self-sealing injection port to serve as an entry point for needles used to inject a filling material into the cavity. Non-limiting examples of material useful for making the injection port include silicone elastomer, such as dimethyl or dimethyl-diphenyl silicone elastomer.

Turning now to FIG. 4, in another embodiment of the invention, a form stable prosthesis 210 is provided. Prosthesis 210 has generally the same features as prosthesis 10, with like elements having like reference numbers increased by 200. Prosthesis 210 comprises a hollow, collapsible shell 208 made up of a rounded or curved anterior portion 211 and a substantially opposing, planar posterior portion 212. A cavity 216 is defined between the anterior portion and the posterior portion. The anterior portion 211 comprises a foam material 214, for example, a form-stabilizing closed-cell, resilient material such as a silicone foam. Like prosthesis 10, the foam material 214 may, in some embodiments, comprise a closed-cell silicone foam and/or a self-skinning foam having a smooth, non porous surface 214a and an opposing textured surface 214b, as described elsewhere herein. In this shown embodiment, prosthesis 210 does not include a separate elastomeric layer on outer surface of anterior portion 211. Instead, foam member 214 includes textured outer surface 214b, skin-side, non-porous inner surface 214a and intermediate closed-cell region 214c therebetween. Posterior portion 212 is secured to foam member 214, for example, at perimeter, using any suitable means.

In this shown embodiment, the foam material 214 has a volume equal to at least about 10%, at least about 20%, or about 30%, or about 50%, or about 60%, or about 70%, or about 80%, or about 90%, or more, of the volume of the cavity 216, when the prosthesis 210 is in the uncollapsed, restored state. In some embodiments, the foam material has a volume greater than the volume of the cavity in the uncollapsed, restored state. In some embodiments, therefore, the foam material 214, in its uncompressed state, may make up at least half of, or most of, the volume of the total implant. In some embodiments, the foam material may make up about 40%, about 50%, about 60%, about 70%, about 80%, or about 90% of the total volume of the implant in its uncompressed state, with a balance being the cavity 216.

For example, the volume of the entire prosthesis 210 may be about 300 cc, the cavity 216 of the implant being about 100 cc and the foam material, in an uncompressed state, having a volume of about 200 cc. As another example, the volume of the entire prosthesis may be about 300 cc, the cavity of the prosthesis being about 50 cc and the foam material, in an uncompressed state, having an volume of about 250 cc. As yet another example, the volume of the entire prosthesis may be about 400 cc, the cavity of the prosthesis being about 200 cc and the foam material, in an uncompressed state, having an volume of about 200 cc. These are provided as examples only, and the present disclosure and claims should not be interpreted as being limited to these particular embodiments.

The current invention can be made using any suitable method available in the art, such as dipping or molding methods. For example, an elastomeric material can be cast onto a mandrel in the shape and form of a breast prosthesis and cured; next, a foam material, such as a silicone foam material, is placed over the portion of the elastomeric material that will constitute the anterior side of the breast prosthesis and then cured. If the foam material is intended as an interior layer, the prosthesis can be flipped inside out when it is taken off of the mandrel. If a self-skinning foam is used, a separate layer of elastomeric material may not be necessary for the anterior portion of the prosthesis; an anterior layer comprising a self-skinning foam material and a posterior layer comprising an elastomeric material can be separately formed, then fused together.

While this invention has been described with respect to various specific examples and embodiments, it is to be understood that the invention is not limited thereto and that it can be variously practiced within the scope of the invention.

What is claimed is:

1. An implantable breast prosthesis comprising:
a hollow, collapsible shell having an expanded configuration, the shell comprising an anterior portion, a posterior portion substantially opposing the anterior portion and a fillable cavity defined between the anterior portion and the posterior portion when the shell is in its expanded configuration;
a form-stabilizing material disposed on or making up a part of at least one of the anterior portion and the posterior portion;
the form-stabilizing material disposed on or forming a part of the shell and providing a resiliency to the shell such that the shell will regain and maintain its expanded configuration in a body cavity after being collapsed during insertion into the body cavity.

2. The prosthesis of claim 1, wherein the form-stabilizing material is closed-cell foam.

3. The prosthesis of claim 1 substantially entirely comprised of silicone.

4. The prosthesis of claim 1, wherein the form-stabilizing material is closed-cell silicone foam.

5. The prosthesis of claim 1 wherein the form-stabilizing material makes up at least a portion of the anterior portion.

6. The prosthesis of claim 1 wherein the anterior portion is rounded and the posterior portion is substantially planar, and the form-stabilizing material is disposed on or forms the rounded anterior portion and is substantially absent on the posterior portion.

7. The prosthesis of claim 1 wherein the foam material has a volume about equal to the volume of the cavity.

8. The prosthesis of claim 1 wherein the form-stabilizing material is a self-skinning foam.

9. An implantable breast prosthesis comprising:
a hollow, collapsible shell having an expanded configuration, the shell comprising an elastomeric anterior portion having a rounded configuration and a substantially opposing elastomeric posterior portion having a substantially planar configuration, and a fillable cavity defined between the anterior portion and the posterior portion when the shell is in its expanded configuration;
a closed-cell foam material disposed on at least one of the anterior portion and the posterior portion;
the foam material providing a resiliency to the shell such that the shell will regain and substantially maintain its expanded configuration in a body cavity after being collapsed for insertion into the body cavity.

10. The prosthesis of claim 9 wherein the foam material is disposed on the anterior portion of the shell and is substantially absent from the posterior portion.

11. The prosthesis of claim 9 wherein the foam material is silicone foam.

12. The prosthesis of claim 9 substantially entirely comprised of silicone.

13. The prosthesis of claim 9 wherein the foam material has a volume about equal to the volume of the cavity.

14. The prosthesis of claim 9 wherein the foam material has a volume greater than the volume of the cavity.
